(19) United States
(12) Patent Application Publication
(10) Pub. No.: US 2019/0350834 A1
(43) Pub. Date: Nov. 21, 2019

(54) MASCARA COMPOSITION AND PACKAGING

(71) Applicant: LVMH RECHERCHE, Saint Jean de Braye (FR)

(72) Inventors: Yohann BICHON, Maisons-Alfort (FR); Caroline KOUSSOUROS, Orléans (FR)

(21) Appl. No.: 16/474,494

(22) PCT Filed: Dec. 27, 2017
(86) PCT No.: PCT/FR2017/053851

§ 371 Date: Jun. 27, 2019

(30) Foreign Application Priority Data
Dec. 30, 2016 (FR) 1663569

(51) Int. Cl. A61K 8/891; A61K 8/87; A61K 8/02; A61Q 1/10

(52) U.S. Cl. CPC A61K 8/891; A61Q 1/10; A61K 8/025; A61K 8/87

(57) ABSTRACT

The invention relates to a mascara with a high wax content containing an association of elastic polyurethane microspheres and non-compressible and non-porous polymethylsilsesquioxane microspheres. The mascara is packaged in a container provided with flexible parts that can be deformed by manual pressure so as to mix the formula. The invention also relates to a method for applying make-up to eyelashes, using said composition and packaging.

The present patent application relates to the field of making up or caring for the eyelashes, and ore specifically relates to a mascara composition containing a high proportion of waxes. This composition comprises at least one liquid phase in which are dispersed waxes, solid particles of at least one first polymer, solid particles of at least one second polymer, and pigments.

The invention also relates to an assembly comprising a mascara composition and a packaging.

### PRIOR ART

Many cream mascaras and waterproof mascaras are sought to give the eyelashes volume by thickening them. It is by adjusting the amount of solid compounds, notably of waxes, that the formulator can improve the makeup result by depositing more material on the eyelashes.

It is, however, difficult to obtain a thickening or volumizing effect without causing the eyelashes to clump together. Specifically, a high solids content leads to a viscous texture which gives rise to lumps of product during application, and which necessitates brushing of the eyelashes several times or several successive applications in order to better spread the product.

It is thus desirable to propose a volumizing mascara which can be applied smoothly and uniformly, without any lumps of product.

It is also desirable to propose a mascara whose volumizing properties are improved from the very first application and whose staying power on the eyelashes is prolonged throughout the day. The absence of running or of overspilling of the mascara onto the contour of the eyes, and also limited crumbling of the product coating the eyelashes, are desired.

In a cream mascara, just as in a waterproof mascara, the waxes form a structuring network which provides consistency to the liquid in which they are dispersed. This network of wax crystals has a tendency to become irreversibly embrittled over time, which reduces the quality of the makeup in the gradual course of use. A need consequently remains to provide a mascara whose structure is stable in the packaging over time, so as to conserve an optimum makeup result throughout the use of the product.

A need also remains to propose a mascara in which the waxes are uniformly dispersed in the packaging, remain regularly dispersed throughout the duration of use of the product, and are in large amount to obtain a heavy deposit on the eyelashes.

It has now been discovered that a combination of two particular polymers in a composition comprising high contents of waxes makes it possible to obtain the mascara whose volumizing effect is improved and whose texture is fluid and creamy at the time of application. This combination makes it possible to slow down the degradation of the network of wax crystals over time. Finally, each of these polymers participates in more substantial loading of the mascara brush, when the composition is placed in a tube that can be deformed by hand pressure, for example.

### DESCRIPTION OF THE INVENTION

Thus, the present invention describes a composition for making up or caring for the eyelashes, comprising a liquid, such as water or an oil, in which are dispersed waxes, solid particles of at least one first polymer, solid particles of at least one second polymer, and pigments, the first polymer being chosen from crosslinked polyurethanes, and the second polymer being chosen from polymethylsilsesquioxanes and crosslinked polymethyl methacrylates.

The invention also describes a makeup process comprising a makeup composition placed in a packaging and application assembly having a flexible wall which can be deformed by exerting hand pressure, said composition comprising a liquid in which are dispersed waxes and solid particles of at least one first polymer, the polymer being chosen from crosslinked polyurethanes, polymethylsilsesquioxanes, crosslinked polymethyl methacrylates, and mixtures thereof.

The invention also describes a process for making up the eyelashes, which comprises at least the following steps:
providing a user with a mascara composition with a high wax content containing a polymer chosen from crosslinked polyurethanes, polymethylsilsesquioxanes, crosslinked polymethyl methacrylates and mixtures thereof, said composition being packaged in a packaging and application assembly comprising i) a container equipped with a draining means and with a flexible part on at least one of its internal or external surfaces and ii) a stopper to which is securely fastened an application brush which is immersed in the composition,
pressing of said flexible part by the user so as to blend the composition contained in the container,
opening the tube and draining the brush covered with composition by removing the stopper, and
brushing the eyelashes.

The particular combination of the two polymers, or the particular combination of one polymer and of a conditioning assembly, advantageously makes it possible to soften a formula just before it is applied to the eyelashes. This softening may be obtained by exerting pressure on a deformable part of the packaging. The mascara, which has a high viscosity due to the high concentration of waxes, may be applied more regularly, may lead to smoother covering of the eyelashes and may allow greater thickening of the eyelashes from the very first application.

The combination of the two polymers, or the combination of one polymer and of a packaging assembly, proposed in the context of the invention, also makes it possible to restructure the wax network, so as to increase the volumizing effect on each application and throughout the service life of the product. The volumizing effect may also be prolonged throughout the day over a longer period than for the mascaras of the prior art.

The present invention also describes a composition for making up or caring for the eyelashes, comprising a liquid, waxes, solid particles of at least one first polymer, solid particles of at least one second polymer different from the first one, and pigments, the particles of the first polymer being elastic, and the particles of the second polymer having very low oil-absorbing and water-absorbing properties.

This composition may more specifically comprise a combination of first solid particles which deform under a mechanical stress and which at least partially regain their shape after the stress is removed, and second solid particles, different from the first ones, which are non-porous and which have no affinity for the liquid dispersion medium, which conserve their shape irrespective of the mechanical stress exerted.

These two categories of starting materials participate in the blending of the composition on each use of the product by the user, either by a pressure exerted on the packaging in which the composition is contained, or by shear stress of the brush loaded with product at the time of its draining or of its application.

This blending has several consequences, such as maintaining the structure of the wax network throughout the duration of storage and of use of the product, easier application of the product to the eyelashes, and the obtention of an immediate and substantial volumizing makeup result.

In addition, the product has a creamy texture without detracting from the application properties. It is thick without being heavy and provides a lot of material.

### DETAILED DESCRIPTION OF THE INVENTION

Thus, in a first aspect, the invention relates to a mascara composition comprising at least one liquid phase, waxes, solid particles of at least one first polymer, solid particles of at least one second polymer, and pigments,

the liquid phase representing from 30% to 60% by weight, preferably from 40% to 55% by weight,

the waxes representing from 15% to 40% by weight, preferably from 25% to 40% by weight,

the pigments representing from 5% to 15% by weight,

the sum of the amount of the first polymer and of the amount of the second polymer being between 0.01% and 5% by weight,

characterized in that the first polymer is chosen from crosslinked polyurethanes, and in that the second polymer is chosen from polymethylsilsesquioxanes,

the percentages being expressed relative to the weight of the composition.

The texture of the product of the invention affords an improved and furthermore immediate volumizing effect, which is better maintained in the course of the day. The eyelashes are much thicker, longer, well curled, supple, soft and comfortable. The mascara of the invention uniformly covers the eyelashes from the root to the end. The successive depositions of product overlap well from one passage to another, and the loading is satisfactory from the very first brushing.

The present patent application also describes a mascara composition comprising at least one liquid phase, waxes, solid particles of at least one first polymer, solid particles of at least one second polymer, and pigments, the particles of the first polymer being elastic, and the particles of the second polymer having very low oil-absorbing and water-absorbing properties.

The first polymer is advantageously in the form of microspheres which have a quite low hardness (also known as compressibility), which can be measured via various methods. For example, the hardness of the particles of the first polymer may be less than 10 N/mm², by measuring the force required to obtain a 10% deformation of the index K (denoted K(10%)), with a Shimadzu Microcompression Testing Machine MCTM-500 reference compression machine. The hardness of the particles, evaluated by measuring the force required to obtain a 10% deformation of the index K (denoted K(10%)), is advantageously between 0.01 and 1 N/mm², more preferably between 0.1 and 0.5 N/mm². This method also makes it possible to evaluate the percentage of elastic deformation of said particles: this percentage is preferably greater than 10%.

The hardness of the particles of the first polymer, measured by the method ASTM D2240 (Shore hardness), may be less than 10° D, preferably less than 1° D.

The particles of the first polymer are advantageously elastic their elasticity value being able to be determined via various methods known to a person skilled in the art.

The elasticity of the particles, expressed as being the degree of elastic deformation, may be measured with a Shimadzu Microcompression Testing Machine MCTM-500 reference compression machine. The degree of elastic deformation is preferably greater than 5%, more preferably greater than 10%, 15%, 20%, 25%, 30%, or even 35%, notably when the compression force exerted corresponds to K(10%) mentioned previously.

The elasticity of the particles of the first polymer may also be characterized by a modulus of elasticity, measured according to the standard ISO 178 (2010), of less than 1000 MPa, preferably less than 500 MPa, more preferably less than 100 MPa.

The second polymer is preferably in the form of microspheres, the specific surface area of which is less than 30 m²/g, the specific surface area being measured via a method known to a person skilled in the art, notably the standard ISO 9277 (2010).

The particles of the second polymer preferably have very low water-absorbing and oil-absorbing capacities. The oil absorption of the particles of the second polymer may be measured at 25° C. according to a method known to those skilled in the art: it is, for example, from 0 to 0.9 g/1 g of oil under the conditions of the standard ASTM D281-8. According to a particular embodiment of the invention, the oil absorption of the particles of the second polymer, measured at 25° C. according to this method, ranges from 0 to 0.6 g/1 g of oil.

The water absorption of the particles of the second polymer, evaluated at 20° C. by means of the standard DIN 53 472, may be less than 1%. Finally, evaluated at 20° C. by the standard ISO 62 (2008), the water absorption value of the particles may be less than 1% also.

The particles of the first polymer may have oil-absorbing and water-absorbing properties higher or lower than those of the particles of the second polymer.

On the other hand, it is preferred for the particles of the second polymer to have a much higher hardness and much lower elasticity than those of the particles of the first polymer. It is also preferred for the particles of the second polymer to be filled and non-porous.

The product according to the invention has a more or less fluid creamy texture: its viscosity is preferably greater than 150000 mPa·s, and advantageously between 200000 and 600000 mPa·s. The viscosity of the composition may be measured at 25° C. using a Rheomat 180 (from the company LAMY) equipped with an MS-R1, MS-R2, MS-R3, MS-R4 or MS-R5 spindle chosen as a function of the consistency of the composition, rotating at a spin speed of 200 rpm. The measurement is taken after 10 minutes of rotation.

The composition may contain one or more liquid phases, which may be fatty or aqueous.

For example, the composition contains a single liquid phase comprising water or a single liquid phase comprising at least one oil. The composition may be anhydrous, in the sense that water is not added during the process for its manufacture.

The composition may also be an emulsion of two liquid phases, including water and a liquid fatty phase comprising oils. In this case, the composition may be a water-in-oil emulsion or an oil-in-water emulsion.

Besides one or more liquid phases, the composition may also contain at least one other phase which is solid and dispersed in at least one of the liquid phases. The composition is, for example, a dispersion of solid particles in an aqueous liquid phase, or in an oily liquid phase.

The oils may be chosen from volatile or nonvolatile dimethicones, cyclomethicones, isododecane, isohexadecane or isodecane, liquid paraffin, hydrogenated polydecene, propylene carbonate, isotridecyl isononanoate, isostearyl neopentanoate, tridecyl neopentanoate (cetyl octanoate), nonvolatile silicone oils, notably phenyl trimethicones. It is preferred for the liquid fatty phase to predominantly or even exclusively comprise volatile oils in the form of a gel, such as volatile dimethicones, cyclomethicones, isododecane, isohexadecane or isodecane, gelled with an organomodified clay.

The liquid phase or the mixture of liquid phases represents, for example, from 30% to 60% by mass, preferably from 40% to 55% by mass relative to the mass of the mascara composition. The liquid phase is preferably water or isododecane. For example, water or isododecane represents from 30% to 60% by mass, preferably from 40% to 55% by mass, relative to the mass of the mascara composition.

According to one embodiment of the invention, the liquid represents from 40% to 55% by weight, the waxes represent from 20% to 25% by weight and the pigments represent from 8% to 12% by weight.

The first polymer may be in the form of particles consisting of one or more polyurethane polymers, or in the form of composite particles comprising an organic or mineral non-polyurethane core and a polyurethane shell. The polyurethane may be a polyurethane polymer or a copolymer partly comprising urethane units.

It is preferred for the polyurethanes included in the composition of the particles of the first polymer to be crosslinked.

The particles of the first polymer consist, for example, of a polyurethane-coated polymethyl methacrylate core, or of a polyurethane-coated silica core. The particles of the first polymer may also consist of polyurethane.

It is preferred for the particles of the first polymer to have elasticity properties. Thus, it is preferred for the first polymer not to be a urethane-acrylate copolymer whose elasticity is zero.

In composite powders with a hard core and a polyurethane coating, the relative amount of the core and of the coating will be adjusted so as to obtain satisfactory elasticity and hardness.

For the purposes of the invention, the polyurethane is not introduced into the composition in the form of an aqueous dispersion of anionic polyurethane, such as the polyurethane lattices commonly used as film-forming polymer in the formulation of mascaras.

The first polymer may be in the form of microspheres of a crosslinked polyurethane obtained by copolymerization of an aliphatic diisocyanate (typically of a molecular weight of between 100 and 300 g·mol⁻¹), which is preferably linear, of an aliphatic triol, preferably of low molecular weight (typically between 100 and 150 g·mol⁻¹) and optionally of a lactone. Use may be made, for example, of a polymer whose INCI name is HDI/trimethylol hexyllactone crosspolymer as first polymer. Such a polymer is sold under the brand name Plastic Powder® D-400.

A mole percentage between the diisocyanate monomer and the triol monomer will be used so as to obtain the desired compressibility properties. The polyurethane particles are preferably manufactured by polycondensation of the monomers mentioned previously in a lipophilic solvent.

The percentage of particles of the first polymer, preferably of crosslinked polyurethane, is, for example, between 0.01% and 5% by weight, preferably between 0.05% and 2% by weight, between 0.08% and 1.5% by weight, between 0.1% and 1.5% by weight, or even between 0.2% and 1.0% or between 0.2% and 0.5% by weight.

The particles of the first polymer have a mean size ranging from 1 to 40 microns, preferably from 1 to 25 microns. They are preferably in the form of microspheres.

The particles of the second polymer have a mean size ranging from 1 to 40 microns, preferably from 1 to 25 microns. They are preferably in the form of microspheres.

The particles of the first polymer and the particles of the second polymer preferably have a mean size ranging from 1 to 25 microns.

The polyurethane is preferably of lipophilic nature and dispersed in the waxes of the composition.

The second polymer may be in the form of particles consisting of one or more polymethylsilsesquioxane polymers, or in the form of composite particles comprising a non-polymethylsilsesquioxane core (such as an acrylic polymer) and a polyimethylsilsesquioxane shell.

According to one embodiment, the second polymer is a crosslinked, preferably a highly crosslinked, polymethylsilsesquioxane.

The polymethylsilsesquioxane is preferably lipophilic, and does not have any amphiphilic or emulsifying properties. Examples of polymethylsilsesquioxane amphiphilic silicone resins of brand name Silderm Emulsifying® BG or Silderm Emulsifying® CS therefore do not form part of the present invention. For the purposes of the invention, the term “lipophilic” refers to a polymer which is compatible with the fatty or lipophilic ingredients of the composition, such as waxes.

The polymethylsilsesquioxane of lipophilic nature is thus advantageously dispersed in the waxes. This is likewise the case for the first polymer.

The particles of the second polymer advantageously have a very low capacity for absorbing liquids such as oils and water, so as to maintain their intrinsic hardness once they have been introduced into the composition, and to blend the other ingredients of the composition, notably the waxes, under the effect of a mechanical stress. The values of the oil-absorbing and water-absorbing capacities of the particles of the second polymer, and the method for measuring same, may be in accordance with the description that has been given previously.

For example, the particles of the second polymer have an oil absorption measured according to the standard ASTM D281-84 of less than 1 g/1 g, more preferably less than 0.60 g/1 g.

The second polymer may correspond to the commercial products Tospearl®, which are in the form of hydrophobic microspheres with a mean size ranging from 2.0 to 6.0 microns.

The percentage of particles of the second polymer, preferably of polymethylsilsequioxane, is, for example, between 0.01% and 5% by weight, preferably between 0.05% and 2% by weight, between 0.08% and 1.5% by weight, between 0.1% and 1.5% by weight, or even between 0.2% and 1.0% or between 0.2% and 0.5% by weight.

The sum of the amount of the first polymer and of the amount of the second polymer is, for example, between 0.01% and 5% by weight, preferably between 0.05% and 2% by weight, between 0.08% and 1.5%, between 0.1% and 1.5% by weight, or even between 0.2% and 1.0% or between 0.2% and 0.5% by weight.

The mass ratio between the particles of the first polymer and the particles of the second polymer may be between 1/5 and 5/1, between 1/2 and 4/1, preferably ranging from 1/2 to 3/1.

The waxes are known to those skilled in the art and may be chosen from beeswax, carnauba wax, candelilla wax, hydrogenated jojoba wax, hydrogenated palm oil, rice bran wax, ceresin wax, montan wax, ozocerite waxes, microcrystalline waxes, polyethylene waxes, alcohol-polyethylene waxes, solid fatty alcohols such as stearyl alcohol and behenyl alcohol, polyalkylene glycol derivatives of the waxes mentioned previously such as polyoxyethylenated derivatives (PEG 6-20) of beeswax or of carnauba wax, solid fatty acid triglycerides, and mixtures thereof.

As understood by a person skilled in the art, a wax is a compound of solid appearance at 25° C. which comprises crystalline phases. Its melting point may be defined as the melting point corresponding to the start of melting of this compound or the temperature corresponding to the endothermic peak of the compound, these magnitudes being able to be measured by DSC (Differential Scanning calorimetry) according to the standard ISO 11357-3. The melting point of a wax is preferably greater than or equal to 30° C.

Any wax known to a person skilled in the art of mascara formulation may be used.

The wax used for manufacturing the mascara may be mixed with the other ingredients of the composition in the form of a microdispersion in water or in the form of powder or pellets.

The waxes represent from 18% to 40% by mass, preferably from 25% to 35% by mass, more preferably from 28% to 32% by mass, relative to the mass of the composition.

The mascara composition comprises pigments representing from 5% to 15% by weight, for example from 8% to 12% by mass of the composition.

The pigments are preferably chosen from titanium dioxide, zinc oxides, iron oxides (black, yellow or red), manganese violet, ultramarine blue, chromium hydrate and ferric blue, metal powders such as aluminum powder or copper powder. Among the organic pigments, mention may be made of carbon black, pigments of D & C type, and lakes. Mention may also be made of reflective pigments and nacreous pigments.

The composition may contain at least one film-forming polymer intended to increase the coating of the eyelashes. The amount of film-forming polymer may range from 0.1% to 12%, preferably from 5% to 10% and better still from 6% to 8% by weight, relative to the total weight of the composition.

Among the film-forming polymers that may be included in the composition of the invention are polybutene, ethylcellulose and vinylpyrrolidone (VP) copolymers such as VP/eicosene or VP/hexadecene, and mixtures thereof.

Among the film-forming polymers, mention may also be made of cellulose polymers such as hydroxyethylcellulose, vinyl polymers such as polyvinylpyrrolidones, gum arabics, guar gum, xanthan derivatives, and mixtures thereof.

The compositions may contain adjuvants that are common in the cosmetics field, such as surfactants, hydrophilic or lipophilic gelling agents, hydrophilic or lipophilic active agents, fatty compounds in paste form, preserving agents, antioxidants, pH regulators and fragrances.

The surfactants preferentially used in the composition according to the invention are chosen, for example, from oxyethylenated and/or oxypropylenated ethers, fatty acid esters of polyethylene glycol, polyethylene glycol/polypropylene glycol polycondensates, fatty acid esters and ethers of sucrose or of sorbitan, fatty acid esters of glycerol or of sorbitol, such as glyceryl stearate, and fatty acid salts such as triethanolamine stearate. In the context of the present invention, use will preferably be made of triethanolamine stearate.

The present patent application describes an assembly for packaging and applying a mascara which may or may not contain one of the compositions described previously. Said assembly comprises:
a hollow container intended to contain the mascara and including the wall defining an inner space and an aperture, at least one first part of the wall being rigid, and at least one second part of the wall being more flexible than the first rigid part and being able to be moved from an expanded position to a forced position,
closing means intended to removably close off said aperture, and
an applicator head borne on a stem that is securely fastened to the closing means, said applicating head being mounted so as to be, in the closed position of the closing means, able to be placed in contact with the mascara inside said hollow container.

By exerting a pressure on the second flexible part of the wall, a user can blend the mascara, which is generally thixotropic, so as to break its resting viscosity and to ensure efficient fluidization thereof, and thus satisfactory loading of the mascara brush and better distribution of the mascara in the container.

It is understood that the terms “rigid” and “flexible” are understood relative to each other, i.e. the first rigid part is more rigid than the second flexible part and that the second flexible part is more flexible than the first rigid part. In addition, a rigid part is a part configured to remain substantially at rest and to conserve its shape when the user exerts manual pressure on this part. For example, the deformation of the rigid part when a manual pressure is exerted thereon may be invisible to the naked eye. A deformation may be considered as invisible to the naked eye if it is less than half a millimeter. The manual pressure may be a pressure that can be exerted, for example, by a user's fingers, notably by two opposite fingers.

A flexible part is a part configured to deform substantially, notably visibly to the naked eye, when a manual pressure is exerted thereon. Thus, the expanded position and the forced position may be distinguished by the naked eye. Preferably, the expanded position and the forced position are separated, at at least one point, by a distance at least equal to half a millimeter.

Optionally, the first rigid part has at least one window that is closed off by the second flexible part. The wall is thus flexible at the window. The user thus readily knows where she can exert pressure to obtain the blending effect explained previously.

Optionally, the first rigid part delimits the window axially and circumferentially. It is understood that the window is entirely surrounded by the first rigid part, which ensures that the container maintains its shape despite the possible deformations of the second flexible part of the wall.

Optionally, the stem is configured so that, in the closed position of the closing means, the applicator head is at least partly facing, axially, the window. In other words, in said closed position, the applicator head is positioned axially so as to be facing the window. By means of these provisions, the blending effect may be obtained directly in the region of the applicator head. Furthermore, the pressure exerted on the second flexible part may allow the user to directly deposit product onto the applicator. Preferably, in the closed position of the closing means, the applicator may also be entirely axially facing the window.

The first rigid part is assembled onto the second rigid part in a leaktight manner so as to form a leaktight wall for the container. According to one example, the second flexible part may be heat-sealed onto the first rigid part.

Optionally, the wall comprises a tubular body forming the first rigid part.

Optionally, the wall comprises a membrane forming the second flexible part.

Optionally, the membrane is assembled onto the tubular body radially to the exterior of the tubular body. In certain embodiments, the second flexible part may form the exterior of the wall. These provisions facilitate the manufacture of the container and improve its appearance.

Optionally, the tubular body comprises a ring and a skirt that is radially recessed relative to said ring. The skirt may extend radially from the ring. According to one example, the ring and the skirt may be connected via a frustoconical portion and/or a shoulder. Where appropriate, the window may be provided in the skirt. The membrane may be placed in the axial extension of the ring, radially to the exterior of the skirt.

Optionally, the assembly also comprises at least one neck securely fastened to said wall.

Optionally, the assembly also comprises at least one draining means securely fastened to said neck.

Optionally, said applicator head is a flocked quill, a felt, a coarse brush or a fine brush.

Optionally, the first rigid part comprises a material chosen from the group formed by polyethylenes such as low-density polyethylenes, high-density polyethylenes and mixtures of polyethylenes of different densities; polyoxymethylenes; polypropylene; poly(vinyl chloride); polyesters; polyamides; Nylons; and mixtures of other plastics such as mixtures of polycarbonate and of polypropylene; and mixtures thereof. Preferably, said material is polyoxymethylene.

Optionally, the second flexible part comprises an elastomeric or thermoplastic material, such as a silicone, a fluorosilicone, a butyl rubber, a nitrile rubber, VITON, an ethylene-vinyl acetate (EVA) copolymer, a polyether amide block copolymer, a polyester elastomer, an ethylene and propylene rubber, a polyurethane, a styrene-ethylene-styrene (SES), a styrene-butadiene-styrene (SBS), a styrene-isoprene-styrene, a styrene/butylene-ethylene/styrene, a synthetic or nonsynthetic latex, and a rubber, and mixtures thereof.

According to one embodiment, a mascara packaging and application assembly comprises a hollow container intended to contain the mascara, means for closing said container and an applicator head.

The hollow container includes a wall defining an inner space and an aperture, the aperture being in the present case defined by a neck. The wall comprises a first part that is relatively rigid and a second part that is relatively flexible. The relatively rigid first part is formed, in the present case, by a tubular body. In this embodiment, the tubular body extends along an axial direction and successively comprises, in the axial direction, the abovementioned neck, a ring, a skirt and a bottom wall.

In this embodiment, the ring is substantially cylindrical. It is recalled that a cylinder is a surface defined by a straight line of fixed direction (generatrice) describing a closed curve of any shape. In the present case, the ring may be a cylinder frustum with a circular base.

Windows are made in the tubular body, more specifically in the skirt. In this embodiment, two windows are made in the skirt. However, any number of windows may be envisaged. In this embodiment, the windows are diametrically opposite in a radial direction, i.e. in a direction perpendicular to the abovementioned axial direction. Moreover, each window may be of curved or polygonal shape with rounded or non-rounded corners. For example, a window may be of rectangular, oval, oblong or elliptical shape.

In this embodiment, besides the presence of the windows, the skirt is substantially cylindrical, for example with a circular base.

The bottom wall is provided at an axial end of the tubular body, notably of the skirt, on the opposite side to the neck. The bottom wall closes off the tubular body. The bottom wall may extend substantially radially. The bottom wall may be flat, at least on the side toward the interior of the container, so as to avoid the accumulation of mascara in inaccessible zones. The side of the bottom wall that is to the exterior of the container may be flat or may have a desired shape, for example to improve the stability of the container.

Moreover, the relatively flexible second part is formed, in the present case, by a membrane, preferably an elastic membrane. It is understood that the elasticity limit of the membrane is such that the membrane remains within its range of elastic deformation when it is stressed between the expanded position and the forced position.

In the present embodiment, the membrane closes off the windows provided in the tubular body. Thus, at the windows, the wall of the container is reduced at its flexible second part. More particularly, at the windows, the wall of the container consists solely of the membrane.

As indicated previously, the flexible second part is able to be moved from an expanded position to a forced position. This movement of the flexible second part may be affected directly or indirectly. According to one example, the windows are provided larger than an average human finger, at least in one direction, so that a finger can exert a pressure by pressing on the membrane. For example, the windows may extend at least over substantially a centimeter in width. According to another example, the pressure may be exerted on the flexible second part indirectly by means of optionally articulated mobile buttons, which transmit, optionally with a reduction ratio, a force or a movement of the flexible second part.

In this embodiment, notably to facilitate the assembling of the membrane to the tubular body, the membrane is configured to at least partly espouse the exterior shape of the tubular body, and at least partly the portion of the tubular body which comprises the windows. More particularly, in this case, the membrane espouses the shape of the skirt and of the bottom wall, in particular to the exterior thereof. It is understood that the membrane does not comprise an aperture where the windows are provided, so as to ensure the leaktightness of the wall of the container.

In the present case, the membrane is of substantially rotationally symmetric shape. In this example, the membrane comprises a cylindrical frustum and a closing disk provided at the end of the cylindrical frustum. The cylindrical frustum is provided in the axial extension of the ring of the tubular body. In addition, the interior of the cylindrical frustum is in contact with the exterior of the skirt. Thus, the skirt is radially recessed relative to the ring, by a distance substantially equal to the radial thickness of the membrane, notably of the cylindrical frustum. These provisions contribute toward the esthetic effect of the assembly.

To further improve the esthetics of the assembly, the thickness of the membrane may vary, in particular with regard to the windows. According to one example, the flexible second part may be thinned with regard to the windows. Moreover, the second part of the wall may comprise decorative patterns, for example drawn, engraved or printed patterns, or patterns made via any other method known per se.

As indicated previously, the wall forms an assembly that is leaktight per se. Thus, the rigid first part is assembled to the flexible second part in a leaktight manner. More particularly, the tubular body and the membrane are assembled in a leaktight manner, for example heat-sealed together.

The closing means intended to removably close off said aperture may be, for example, a cap to be clipped, screwed or engaged on the container, notably on its neck. Where appropriate, the container or the neck may comprise complementary clipping, screwing or engaging means. Other closing means are known to those skilled in the art. These closing means may comprise sealing means, examples of which are known to those skilled in the art.

As indicated previously, the applicator head is borne on a shaft that is securely fastened to the closing means, said applicator head being mounted so as to be, in the closed position of the closing means, able to be placed in contact with the mascara inside said hollow container.

The present patent application also describes a process for making up the eyelashes, comprising a packaging containing a composition in accordance with one of the aspects of the first subject of the invention described above.

In greater detail, the present invention describes a mascara product comprising:
a mascara composition comprising at least one liquid phase, waxes, solid particles of a polymer, and pigments,

the liquid phase representing from 30% to 60% by weight,

the waxes representing from 15% to 40% by weight, preferably from 20% to 35% by weight,

the pigments representing from 5% to 15% by weight,

the polymer being chosen from polymethylsilsesquioxanes, crosslinked polymethyl methacrylates and crosslinked polyurethanes, and the polymer representing between 0.01% and 5% by weight,

the percentages being expressed relative to the weight of the composition, and
a packaging and application assembly having a flexible wall which can be deformed by exerting hand pressure, so as to bring about blending of said composition it contains.

The packaging may be in accordance with the description that has been made thereof previously.

The ingredients of the composition may also be in accordance with the description that has been made thereof previously.

Another part of the description of the present patent application relates to a process for making up the eyelashes, comprising the application to said eyelashes of the composition according to the present patent application.

This process may consist of a makeup process which comprises at least the following steps:
providing a user with a mascara composition packaged in a packaging and application assembly comprising i) a container equipped with a draining means and a flexible part on at least one of its internal or external surfaces and ii) a stopper to which is securely fastened an applicator brush which is dipped into the composition, said composition being in accordance with one of the descriptions that has been made thereof hereinabove,
pressing by the user on said flexible part so as to blend the composition it contains,
opening the tube and draining the brush covered with composition by removing the stopper, and
brushing the eyelashes.

The brush may be a loading brush known to those skilled in the art, such as a brush of bottle-brush type consisting of Nylon® fibers held by a twisted wire, or a brush obtained by thermoplastic molding of synthetic materials (HDPE, LDPE, silicone derivatives, POM). The brush may be connected to the stopper via a flexible stem.

In the examples below, all the percentages are given on a weight basis relative to the weight of the composition, the temperature is in degrees Celsius and the pressure is atmospheric pressure, unless otherwise indicated.

### EXAMPLE 1

### Mascara Composition

The following mascara was prepared. The mass percentage of the ingredients and the order of incorporation thereof into the composition are detailed in Table 1.

TABLE 1

| | INGREDIENTS (INCI) | WEIGHT % |
|---|---|---|
| A1 | AQUA | Qs 100 |
| | TRIETHANOLAMINE | 2.0 |
| A2 | GUM ARABIC | 2.0 |
| | HYDROXYETHYLCELLULOSE | 0.5 |
| B1 | BEESWAX | 10.0 |
| | PARAFFIN | 8.0 |
| | STEARIC ACID | 4.0 |
| | GLYCERYL STEARATE | 4.0 |
| | CARNAUBA WAX | 5.0 |
| | VP/EICOSENE COPOLYMER | 2.0 |
| | POLYBUTENE | 2.0 |
| B2 | BLACK IRON OXIDE | 8.0 |
| | HDI/TRIMETHYLOL HEXYLLACTONE | 0.3 |
| | CROSSPOLYMER | |
| | POLYMETHYLSILSESQUIOXANE (Tospearl ®) | 0.10 |

Preparation Process

The composition was prepared according to the following process.

Fatty Phase B:

Melt B1 in the main beaker with stirring using a Staro blender on a water bath at 85° C. Once the mixture has melted, add B2.

Liquid Aqueous Phase A:

In a second beaker, weigh out A1, and place it on a water bath at 85° C. with stirring using a Staro blender. Once the mixture is homogeneous, disperse A2 therein with stirring.

Preparation of the Emulsion:

Add the aqueous phase A to the main beaker B at 85° C. over 5 minutes. Stop the heating and allow the formula to cool while gradually reducing the stirring.

Makeup Properties

The product of example 1 of the invention was evaluated by a panel, in a blind test, for a duration of use of one week, once a day, under conventional application conditions, and compared with a reference product. The panel consisted of 59 women from 26 to 45 years old, equitably distributed, who are regular users of mascaras of the select market.

During this test, the properties of the product of the invention were compared with those of a reference product not comprising either the HDI/TRIMETHYLOL HEXYLLACTONE CROSSPOLYMER, or the POLYMETHYLSILSESQUIOXANE. The total amount of the two compounds left out was carried over onto the amount of water, in the reference product.

Here is the summary of the assessments that were collected for the product of the invention. It was confirmed that the average of the numerical values obtained is significant.
Application: the mascara uniformly covers the eyelashes from the root to the end, covering well from one application to another, the eyelash fringe is immediately covered.
Volume effect: the texture is ultra-volumizing, the volumizing effect is immediate and spectacular and remains constant from morning to evening.
Overall makeup result: the eyelashes are well covered, much thicker, lengthened well and curled well.
Makeup result: the satisfactory loading on the first application, it is obtained rapidly, the eyelashes are supple, soft and comfortable.

As regards the composition with the reference product, the consumers found the texture to be more coating (90% versus 76%), the eyelashes more lengthened (86% versus 68%), the persistence of the volume from morning to evening to be better (84% versus 64%), the texture much more volumizing (72% versus 59%), the volume result much more spectacular (62% versus 39%).

### EXAMPLE 2

### Mascara Composition

The following mascara composition was prepared. The mass percentage of the ingredients and the order of incorporation thereof into the composition are detailed in Table 2.

TABLE 2

| | INGREDIENTS (INCI) | WEIGHT % |
|---|---|---|
| A1 | AQUA | QS 100 |
| | AMINOMETHYL PROPANEDIOL | 1.0 |
| A2 | HYDROXYETHYLCELLULOSE | 1.0 |
| A3 | SYNTRAN PE 5760 | 12.0 |
| B1 | PEG-12 CARNAUBA | 9.3 |
| | PEG-8 BEESWAX | 8.1 |
| | STEARIC ACID | 4.5 |
| | PARAFFIN | 3.6 |
| | STEARYL ALCOHOL | 2.9 |
| | VP/EICOSENE COPOLYMER | 2.0 |
| B2 | BLACK IRON OXIDE | 8.0 |
| | POLYMETHYLSILSESQUIOXANE (Tospearl ®) | 0.1 |
| | HDI/TRIMETHYLOL HEXYLLACTONE | 0.1 |
| | CROSSPOLYMER | |

The composition was prepared according to the following process.

Fatty Phase B:

Melt B1 in the main beaker with stirring using a Staro blender on a water bath at 85° C. Once the mixture has melted, add B2.

Liquid Aqueous Phase A:

In a second beaker, weigh out A1, place it on a water bath at 85° C. with stirring using a Staro blender. Once the mixture is homogeneous, disperse A2 therein with stirring. When the mixture is homogeneous, add A3 at 85° C. before emulsion.

Preparation of the Emulsion:

Add the aqueous phase A to the main beaker B at 85° C. over 5 minutes. Stop the heating and allow the formula to cool while gradually reducing the stirring.

### EXAMPLE 3

### Mascara Composition

The following mascara composition was prepared. The mass percentage of the ingredients and the order of incorporation thereof into the composition are detailed in Table 3.

TABLE 3

| | INGREDIENTS (INCI) | WEIGHT % |
|---|---|---|
| A1 | POLYBUTENE | 2.0 |
| | CARNAUBA WAX | 7.5 |
| | BEESWAX | 13.5 |
| | PARAFFIN | 5.0 |
| | VP/EICOSENE COPOLYMER | 2.0 |
| A2 | BLACK IRON OXIDE | 8.0 |
| | POLYMETHYLSILSESQUIOXANE (Tospearl ®) | 0.2 |
| | HDI/TRIMETHYLOL HEXYLLACTONE | 0.2 |
| | CROSSPOLYMER | |
| B | ISODODECANE | QS 100 |
| | DISTEARDIMONIUM HECTORITE | 5.1 |
| | PROPYLENE CARBONATE | 1.5 |
| C | WATER | 5.0 |
| | PHENOXYETHANOL | 0.3 |

The composition was prepared according to the following process:

Preparation of Phase A:

In a main beaker, melt A1 on a water bath (90° C.). When the phase is molten and homogeneous, add A2. Homogenize rapidly while hot and re-immerse the beaker in the water bath (90° C.) until a homogeneous paste is obtained.

Preparation of the Liquid Phase B:

In a secondary beaker, disperse the modified hectorite in the isododecane, without heating and with stirring using a Rayneri blender, When the phase is homogeneous, add the propylene carbonate. Disperse using a Rayneri blender until a homogeneous gel is obtained.
Add phase B to phase A and homogenize rapidly with a spatula. Treat the mixture three times in a three-roll mill until a smooth, glossy formula is obtained.
Add phase C. Homogenize with a Rayneri blender until a smooth, glossy formula is obtained.

1. A mascara composition comprising from 30% to 60% by weight of at least one liquid, from 15% to 40% by weight of waxes and from 5% to 20% by weight of pigments, and also comprising microspheres of a crosslinked polyurethane first polymer and microspheres of a crosslinked polymethylsilsesquioxane second polymer, the sum of the amount of the first polymer and of the amount of the second polymer being between 0.01% and 5% by weight, the percentages being expressed relative to the weight of the composition.

2. The mascara composition as claimed in claim 1, characterized the microspheres have a mean size ranging from 1 to 40 microns.

3. The mascara composition as claimed in claim 1, characterized in that the sum of the amount of the first polymer and of the amount of the second polymer is between 0.1% and 2%.

4. The mascara composition as claimed in claim 1, characterized in that the first polymer is a crosslinked polyurethane obtained by copolymerization of a linear aliphatic diisocyanate, of an aliphatic triol and of a lactone.

5. The mascara composition as claimed in claim 1, characterized in that the liquid phase is water or isododecane.

6. The mascara composition as claimed in claim 1, characterized in that the first polymer and the second polymer are dispersed in the waxes.

7. A mascara product comprising:
a mascara composition as claimed in claim 1, and
a packaging and application assembly, said assembly having a flexible wall which can be deformed by exerting hand pressure, so as to bring about blending of said mascara composition it contains.

8. A makeup process which comprises at least the following steps:
providing a user with a mascara composition packaged in a packaging and application assembly comprising i) a container equipped with a draining means and a flexible part on at least one of its internal or external surfaces and ii) a stopper to which is securely fastened an applicator brush which is dipped into the mascara composition, said mascara composition being in accordance with claim 1.
pressing said flexible part so as to blend the mascara composition it contains,
opening the tube and draining the brush covered with the mascara composition by removing the stopper, and
brushing the user's eyelashes with the brush to deliver the mascara composition on the user's eyelashes.
